(19) United States
(12) Patent Application Publication
(10) Pub. No.: US 2003/0109822 A1
(43) Pub. Date: Jun. 12, 2003

(54) Insertion of sensors into soft tissue

(76) Inventors: Nicholas Barnett, Headington (GB); David Reed Markle, Berwyn, PA (US); William Paterson, High Wycombe (GB)

Correspondence Address: MERCHANT & GOULD PC, P.O. BOX 2903, MINNEAPOLIS, MN, 55402-0903, US

(21) Appl. No.: 10/240,802

(22) PCT Filed: Apr. 12, 2001
(86) PCT No.: PCT/GB01/01662

(51) Int. Cl. A61M 1/00

(52) U.S. Cl. 604/27

(57) ABSTRACT

A sensor device is positioned in soft tissue in the human or animal body, by providing a tubular member (1) containing at least one sensor, the tubular member having a distal end (7) supporting an apertured member (30). The apertured member (30) is connected to a surgical needle (25) by passing a suture (24) through the aperture of the apertured member (30) and the eye of the needle (25), passing the needle (25) through the soft tissue and drawing the suture (24) through the soft tissue to pull the tubular member (1) into the soft tissue. The needle (25) is disconnected from the sensor device by cutting the suture (24) or removing the apertured member (30). In the pulling method of the present invention, the sensor device follows the track of the needle and suture. This reduces tissue damage and also reduces the risk of damaging the sensor as it is positioned.

This invention relates to a procedure for introducing sensor devices into soft tissue and, in particular, provides an elongated sensor device which is designed for this purpose.

Currently, sensors including optical and electro-chemical sensors are typically introduced into a blood vessel or cavity in the body. There are established effective ways of achieving placement of sensors in blood vessels which involves introducing an hollow needle into an artery or a vein, into which the sensor is to be fitted. Having made the incision with the needle, a catheter is introduced and the needle removed. The sensor can then be introduced into the blood vessel through the catheter which can either be left in place or removed, leaving the sensor in the desired position in the blood vessel.

The same technique can be used for introducing sensors into soft tissue. However, in contrast with blood vessels, the pushing of a catheter or sensor into soft tissue causes an injury to the surrounding tissue or damage to the sensor. In the first case, measurements taken from the sensor are often distorted by the insult caused to the tissue and, in the second case, damage to the sensor results in unreliable measurements.

The present invention is concerned with an alternative procedure for introducing sensors into soft tissue, which overcomes the above problems.

The present invention provides a method for introducing a sensor device into soft tissue in the human or animal body, which comprises providing a tubular member containing at least one sensor, the tubular member having a distal end supporting an apertured member, connecting the apertured member and a surgical needle by passing a suture through the aperture of the apertured member and the eye of the needle, passing the needle through the soft tissue and drawing the suture through the soft tissue to pull at least the distal end of the tubular member into the soft tissue, and disconnecting the needle from the sensor device.

The pulling method of the present invention, where the sensor device follows the track of the needle and suture, reduces tissue damage and also reduces the risk of damaging the sensor device as it is positioned in the soft tissue.

Suitably the sensor device is pulled through the soft tissue until the apertured member is wholly or partially clear of the needle exit hole, at which time the needle can be disconnected from the sensor device by cutting away the suture or the apertured member. This leaves the sensor device positioned in the soft tissue so that its sensor(s) can provide data on the condition of the tissue.

The present invention also provides a sensor device for use in soft tissue of the human or animal body, which comprises a tubular member containing at least one sensor, the tubular member having a distal end supporting an apertured member as an attachment means for attachment to a surgical needle, for introducing and locating the device within soft tissue.

Typically, the apertured attachment means comprises a loop which can be attached to the needle by a suture. The loop may be attached directly to one end of the sensor device, or attached or formed on post, wire or filament extending from one end of the sensor device.

In one aspect of the invention, the attachment means is formed from a loop of plastics filament or metal wire which is secured to the distal end of the sensor device. For example, a plastic filament may be looped back on itself and welded to a plastics end cap. In the case of a wire loop, the ends of the wire may be embedded in a plastics end cap.

In another aspect of the invention, the attachment means comprises a plastics filament or metal wire which projects from the distal end of the sensor device and the distal end of the wire includes a loop or an enlarged portion which may be apertured for convenient attachment to a needle by a suture.

The sensor device typically comprises an elongate plastics housing containing sensor elements. Preferably, the sensor device is longitudinally reinforced by means of a wire extending lengthwise thereof and attached to the distal end of the sensor device, e.g. by encapsulation in a plastics end cap. The wire serves the purpose of absorbing the tension applied to the sensor device as it is pulled through the tissue. Advantageously, the wire may be extended through the cap to finish in an end loop or be looped back on itself into the end cap, as indicated above.

The present invention also provides a surgical kit for carrying out the novel procedure of this invention, which is preferably provided as a sterile pack, comprising

(a) a sensor device which comprises a tubular member containing at least one sensor, the tubular member having a distal end supporting an apertured member as an attachment means; and either or both of

(b) a surgical needle;

(c) surgical suture material.

The present invention enables a sensor device to be inserted into soft tissue. Suitable applications include insertion in myocardial muscle for monitoring tissue metabolism in regions of heart tissue peri- and post-operatively; use in skeletal muscle and subcutaneous tissue, for example, for monitoring tissue flaps in plastic surgery or to provide an indicator of regional tissue perfusion peri-operatively in surgical or trauma patients; also use for introducing a sensor into liver or kidney tissue post-transplantation.

The invention will be illustrated, by way of example only, the following particular description of specific embodiments in accordance with the invention.

Referring to the accompanying drawings:

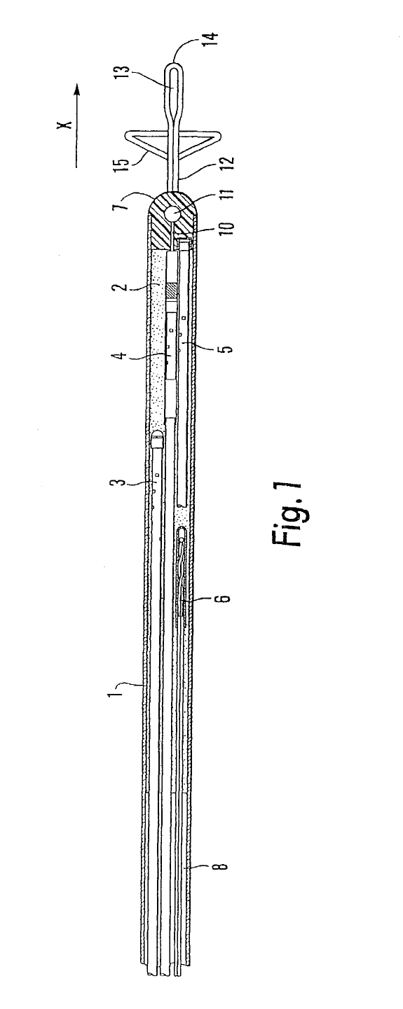

FIG. 1 is a longitudinal section through a composite sensor device in accordance with one embodiment of the invention;

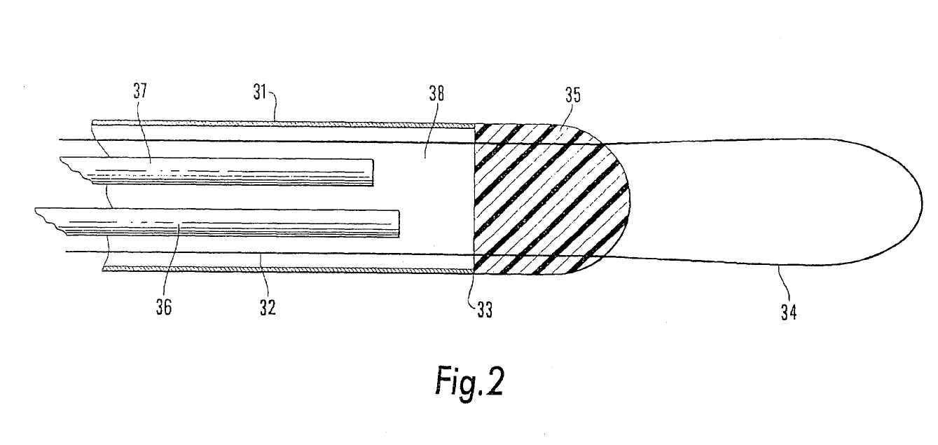

FIG. 2 is a partial longitudinal section through a sensor device in accordance with another embodiment of the invention;

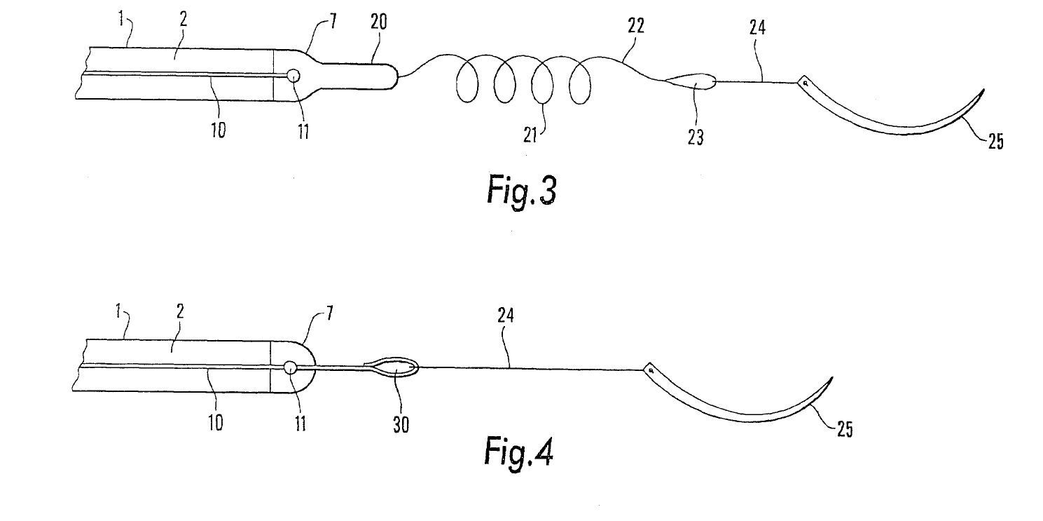

FIG. 3 is a similar view of a further sensor device showing the attachment to a needle for introducing the sensor into tissue;

FIG. 4 is a similar view of another embodiment in accordance with the invention with needle attached.

Referring to FIG. 1, the sensor part of the device comprises an outer sheath (1) which is a micro-porous polyethylene tube which is filled with a hydrophilic hydrogel such as an acrylamide gel (2). Various sensors are supported within the gel. The embodiment shown has multiple sensors, including an oxygen sensor (3) (typically a ruthenium dye in a silicon matrix), a carbon dioxide sensor (4) (typically Phenol Red in a bicarbonate solution), a pH sensor (5) and a thermocouple (6) (typically copper/constantan). The sensor elements are located within about 25 mm of the distal tip (7) of the sensor device, and the micro-porous polyethylene tube (1) is back-filled with a silicone material (8). The distal end of the tube (1) is closed with a polyethylene cap (7). The composite sensor is provided with a longitudinally extending metal wire (10) having an enlarged distal end (11), produced for example by heating the wire tip with a laser, encapsulated in the plastic cap (7). This provides the sensor device with longitudinal strength and enables it to be pulled through soft tissue.

The detailed construction of a suitable sensor device, and its operation for sending signals concerning parameters measured in the body to a remote processor, is described in WO 95/01218, the disclosure of which is incorporated herein by reference.

Welded to the distal end of the cap (7) is a projection (12) formed from a polyethylene monofilament looped back on itself to define a slot (13). On pressing the tip (14) of the projection towards the cap (7), the slot (13) will open for introduction and attachment of a suture, which is attached at its other end to a needle. Projection (12) also supports a moulded anchoring device (15). The purpose of the anchoring device (15) is to maintain the sensor in the desired position within soft tissue of an organ after the sensor has been properly located. When viewed in a direction towards the cap (7), anchoring device (15) comprises a disc-like flap of flexible but resilient plastics material. Alternatively, the anchoring device (15) may comprise a series of radially extending fingers.

The anchoring device (15) is designed so that when the sensor is pulled through tissue in the direction of the arrow X, the disc-like extension or fingers will lie flat, folded in the direction towards the cap (7). However, when traction on the end (14) is stopped, the anchoring device (15) will extend itself outwardly to hold the assembly in place.

FIG. 2 shows a modification of the sensor device of FIG. 1, again having a tubular sensor body (31). A stainless steel wire (32) passes through the tube (31), protrudes from the distal end (33), is turned back on itself to form a loop (34) and then returned into the tube (1). The distal end (33) of the tube (1) is sealed with a molten plastics material which on cooling forms a tip (35) embedding the wire (32). The tubular body (1) is then loaded with sensors (33,37) and a suspending matrix (38), as for the device of FIG. 1.

Within the tube (1), the wire (32) is present as a pair of reinforcing elements. At the distal end of the tube the wire (32) provides a loop (34) securely attached by the tip (35). The loop (34) is used as an attachment point for a suture by which the sensor can be pulled into soft tissue.

FIGS. 3 and 4 show alternative forms of the invention and also the attachment of the sensor device to a suitable surgical needle to pull the sensor into soft tissue. In FIGS. 3 and 4, the same reference numerals are used to indicate equivalent parts.

Referring to FIG. 3, the attachment means and anchoring means differs from that shown in FIG. 1 in the following respects. Cap (7) carries a moulded projection (20) to which is attached an integrally moulded plastics filament (21). A portion of the filament (21) is pre-formed into a coil and its forward end (22) is formed into a loop (23). Loop (23) may be attached by a suture (24) to an insertion needle (25) in a conventional manner. The helical coil (21) provides the same function as anchoring means (15) in FIG. 1. The coil (21) is designed so that when a longitudinal tensile force is applied via the suture (24), the coil (21) will extend as a monofilament thread.

In the arrangement shown both in FIGS. 1, 2 and 3, the needle (25) is introduced into tissue and when the needle emerges from an exit face of the tissue, the sensor can be pulled through by the suture connection to loop (13), (34) or (23) until the tip end containing the sensor elements is contained within the tissue. When the loop (23) or (13) is seen to emerge from the tissue, the loop or suture can be cut off from the sensor device, thereby leaving the sensor properly retained within the tissue. The sensor device is subsequently withdrawn from the soft tissue, after data collection, by pulling from the proximal end.

FIG. 4 shows a further modification of the device in accordance with the invention, in which reinforcing wire (10) is extended through the cap (7) and is formed with a projecting looped end (30) having a through hole for attachment of a suture (24) to a needle (25). The diameter of the polyethylene tube (1) is typically of the order of 0.5 mm in diameter, and the reinforcing wire (10) may be about 0.1 mm in diameter, and preferably a stainless steel wire. The diameter of the projecting part (12) in FIG. 1 will be less than the diameter of the cap (7) and the dimensions of the corresponding parts in FIGS. 3 and 4 will be similar.

Although the above description refers to the cap (7) and parts of the attachment device in FIGS. 1 and 3 as being made from polyethylene, it will be appreciated that other physiologically acceptable materials may be employed, e.g. other polyolefins, nylons or cyanoacrylate. Preferably, the material used for the cap and/or for the attachment means is formed from a thermoplastic material, since this facilitates connecting the attachment parts to the cap by welding or embedding.

1. A sensor device for use in soft tissue of the human or animal body, which comprises a tubular member (1) containing at least one sensor (3), the tubular member (1) having a distal end (7) supporting an apertured member (12) as an attachment means for attachment to a surgical needle, for introducing and locating the device within soft tissue.

2. A device according to claim 1 wherein the attachment means comprises a loop (14,23,30,34).

3. A device according to claim 1 or 2 wherein the attachment means comprises a wire (10,32) which extends lengthwise of the sensor device and projects from the distal end of the sensor.

4. A device according to claim 2 wherein the attachment means comprises a wire (32) which extends lengthwise of the sensor device, projects from the distal end of the sensor, and turns through substantially 360° to return into the tubular body, thus forming a loop (34) external of the tubular body.

5. A device according to claim 2 wherein the attachment means comprises a loop (14) of plastics material which is secured to the distal end (7) of the sensor device.

6. A device according to claim 2 wherein the attachment means comprises a loop (23) of plastics material which is secured to a plastics filament (22) which is secured to the distal end (7) of the sensor device.

7. A device according to claim 5 or 6 wherein the loop or the filament is welded to a plastics cap (7) comprising the distal end of the sensor device.

8. A device according to claim 7 wherein the plastics cap (7) is secured to the tubular member (1) by a wire (10) extending longitudinally within the tubular member (1) and having an enlarged distal end (11) which is embedded in the plastics cap (7).

9. A surgical kit comprising
(a) a sensor device for use in soft tissue of the human or animal body, which comprises a tubular member containing at least one sensor, the tubular member having a distal end supporting an apertured member as an attachment means; and at least one of
(b) a surgical needle;
(c) surgical suture material.

10. A surgical kit according to claim 9, in the form of a sterile pack.

11. A method for introducing a sensor device into soft tissue in the human or animal body, which comprises providing a tubular member (1) containing at least one sensor, the tubular member (1) having a distal end (7) supporting an apertured member (30), connecting the apertured member (30) and a surgical needle (25) by passing a suture (24) through the aperture of the apertured member (30) and the eye of the needle (25), passing the needle (25) through the soft tissue and drawing the suture (24) through the soft tissue to pull at least the distal end (7) of the tubular member (1) into the soft tissue, and disconnecting the needle (25) from the sensor device.
